(19) United States
(12) Patent Application Publication
(10) Pub. No.: US 2010/0163432 A1
(43) Pub. Date: Jul. 1, 2010

(54) MICROSENSOR FOR DETECTION OF D-AMINO-ACIDS

(75) Inventors: Stéphane Marinesco, Lyon (FR); Pierre Pernot, Orsay (FR); Jean-Pierre Mothet, Paris (FR); Raymond Cespuglio, Millery (FR); Oleg Scuvailo, Lyon (FR); Alexey Soldatkin, Kiev (UA); Loredano Pollegioni, Milano (IT); Mirella Pilone, Milano (IT)

Correspondence Address: ARENT FOX LLP, 1050 CONNECTICUT AVENUE, N.W., SUITE 400, WASHINGTON, DC, 20036, US

(73) Assignees: Centre National De La Recherche Scientifque CNRS; Universite Claude Bernard Lyon 1; Universite Paris XI; Universite D'Insubria; Institute of Molecular Biology and Genetics

(21) Appl. No.: 12/443,100

(22) PCT Filed: Sep. 27, 2007
(86) PCT No.: PCT/IB07/02864

§ 371 Date: Mar. 10, 2010

(30) Foreign Application Priority Data
Sep. 27, 2006 (EP) 06291523.6

(51) Int. Cl. G01N 27/26; B05D 5/12; G01N 33/50

(52) U.S. Cl. 205/787; 427/58; 204/403.14; 204/403.1

(57) ABSTRACT

The instant invention is about a microelectrode, in particular for measuring the concentration of a D-amino acid, said microelectrode comprising means for oxidation of said D-amino acid into at least a compound B, means for optimising the detection of said compound B, and means for reducing interferences, in particular interferences due to the oxidation of other species than compound B; a device comprising such a microelectrode; a method for making such a microelectrode, a method for detecting and/or measuring the concentration of a D-amino-acid in a medium.

Microsensor for detection of D-Amino-Acids

The instant invention relates to the field of microsensors or sensing microdevices.

More precisely the invention concerns a microsensor or microelectrode for measurement of D-serine and an electrochemical method for detecting and/or measuring D-amino acid, in particular D-serine, more specifically in vitro, ex vivo and/or in vivo.

There is specially a need for D-amino-acid sensors that can be used for in vivo measurement. For example, for D-serine, which has been recently shown to be present in the Central Nervous System (CNS), in the cortex, hippocampus or developing cerebellum.

This D-amino acid has been recently implicated in several pathologies such as schizophrenia, Alzheimer disease, chronic pain or cerebral ischemia. There is thus a need for pharmacological agents able to interfere with synthesis, release, catabolism and/or uptake system of D-serine in the CNS, as well as for reliable methods for detecting D-serine in vivo and in vitro.

A known method to measure extracellular concentration of D-serine is microdialysis. This method is heavy, expensive and difficult to employ. Moreover, it involves the use of relatively large probes, which may provoke lesions that can compromise the measurement.

Furthermore the principle of this method by itself, comprising the steps of dialysing the extracellular medium, collecting and analysing its content, can induce a perturbation in the physiological functionality of the sample due to the circulation of exogenous extracellular fluid, which may change the local concentration of D-serine as well as of other small metabolites.

Electrochemical methods to detect D-serine are also known, for example in food industry. These methods use sensors having a millimetric or centimetric size, which is generally not compatible with an in vivo use.

The field of microsensors is of increasing interest. There is thus a general need for reliable, cheap, small, precise, selective and/or versatile sensors and more particularly for sensors which can be used in vivo and/or allowing to measure in real time the changes in the concentration of a compound.

As discussed above, there is a special need for a device for detecting D-amino-acids, and in particular D-serine, in vivo and/or allowing to measure in real time its concentration, for example, in order to develop a method to find pharmacological agents able to interfere with synthesis, release and/or elimination of D-amino-acids, and more specifically D-serine in the central nervous system (CNS).

Following a first aspect, the subject matter of the invention is a microelectrode, in particular for measuring the concentration of a D-amino acids, said microelectrode comprising:

means for oxidation of said D-amino acids into at least a compound B,

means for optimising the detection of said compound B, and

means for reducing interferences, in particular interferences due to the oxidation of other species than compound B.

The microelectrode of the invention may have a detection limit of 300 nM or less of the D-amino-acid of interest, in particular D-serine.

The microelectrode presents a good selectivity. For example, solutions of serotonin, dopamine, L-serine and glycine at 10 μM (concentrations much higher than the physiological concentrations of these molecules), do not generate signals bigger than 5% of the ones detected in the same concentration of D-serine.

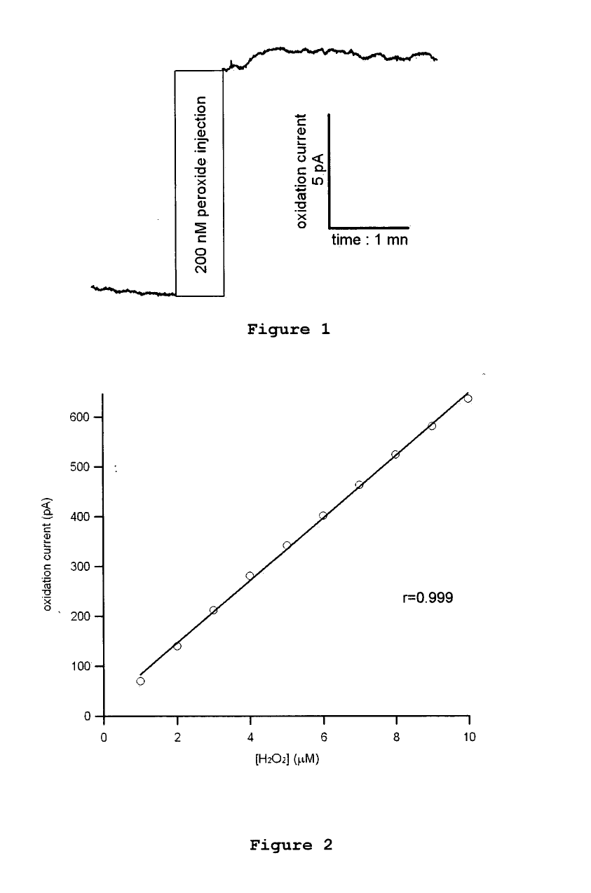

FIG. 1 shows an example experiment where 200 nM of hydrogen peroxide was injected in the recording chamber. The injection produces a step in the oxidation current at the working electrode.

FIG. 2 shows the calibration of the microelectrode of the invention in increasing concentrations of hydrogen peroxide. The microelectrode's response is linear with concentration between 10 nM and at least 10 μM of hydrogen peroxide.

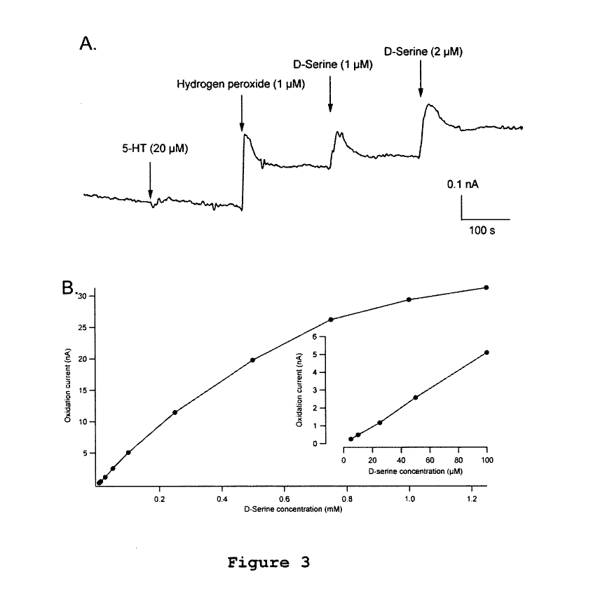

FIGS. 3A and B shows the detection of D-serine using the microelectrode of the present invention.

The FIG. 3A shows the recording oxidation (nA) current at microelectrode of the invention upon time (s) and application of serotonin (5-HT, 20 μM), hydrogen peroxide (H₂O₂, 1 μM), and D-serine (1 μM and 2 μM). D-serine and H₂O₂ are detected as a step in oxidation current, and interference from 5-HT is rejected.

The FIG. 3B shows the calibration curve of a microelectrode of the present invention in D-serine concentrations ranging from 5 μM to 1.2 mM.

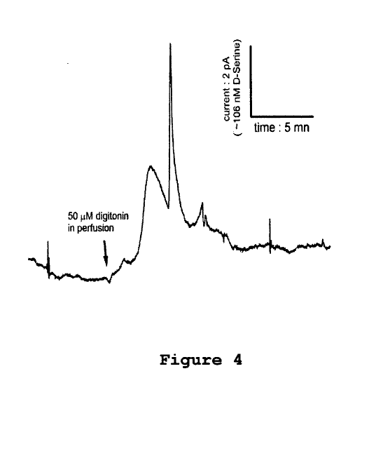

FIG. 4 shows an example of release of D-serine by astrocytes in culture after application of digitonin. Digitonin 50 μM was added to cells in culture. The microelectrode of the present invention, placed above the cells detects the release of D-serine due to the disruption of the astrocytes cellular membrane.

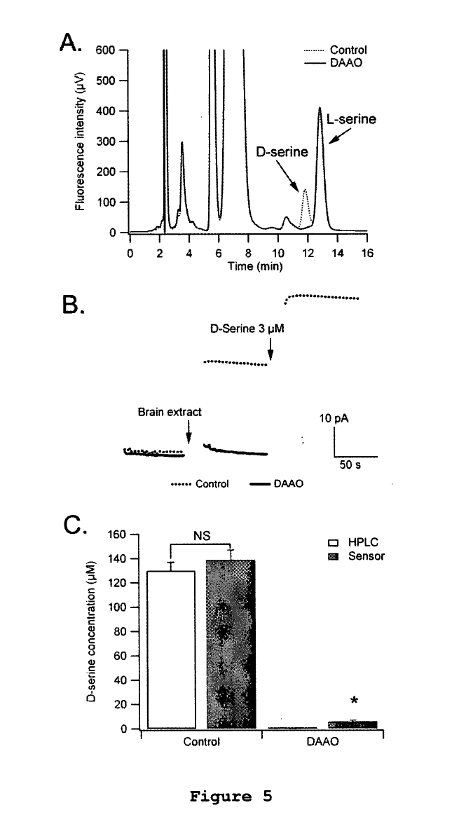

FIGS. 5A, B and C show the excellent selectivity of the microelectorde of the present invention.

FIG. 5A is a typical chromatogram showing a curve of fluorescence intensity (μV) versus time (min). It shows an example D-serine peak after 12 min elution time and its disappearance after incubation with Rhodotorula gracilis D-Amino Acid Oxidase (RgDAAO) and catalase.

The FIG. 5B shows an example of current response recorded at the microelectrode of the present invention upon addition of a brain extract (40× dilution) and 3 μM D-serine (oxidation current in pA versus time in s). The current response to a brain extract preincubated in RgDAAO and catalase was greatly reduced compared to that of a control brain extract. The FIG. 5C is a diagram showing summary results of the comparison between D-serine concentration estimated by HPLC and the microelectrode of the present invention.

By “microelectrode” in the present invention is meant a small size electrode, in particular an electrode having a mean diameter of less than 1 mm, specially of less than 500 μm, more particularly of less than 250 μm, especially of less than 150 μm and more specifically less than 100 μm, or even less than 50 μm.

The definition of a “microelectrode” according to the International Union of Pure and Applied Chemistry is as follows: “microelectrode is any electrode whose characteristic dimension is, under given experimental conditions, comparable or smaller than the diffusion layer thickness.”Stulik K, Amatore C, Holub K, Marecek V and Kutner W (2000) Microelectrodes. Definitions, characterization and applications (technical report). Pure Appl. Chem. 72: 1483-92.[1]

The means for oxidation of the D-amino acids may be at least one D-amino-acid oxidase (DAAO), in particular a D-amino-acid oxidase which leads to the production of hydrogen peroxide as compound B.

A D-amino-Acid Oxidase (DAAO) may be flavoenzyme that contains a molecule of covalently or non-covalently bound flavin adenine dinucleotide as cofactor, which is the site of redox reaction

More precisely, the means for oxidation of the D-amino acid may be at least one D-amino-acid oxidase (DAAO) which doesn't need the addition of a cofactor in the medium to oxidise the D-amino-acids. The DAAO may have a natural or artificial cofactor that is tightly bound to the apoprotein moiety (i.e., no inactive apoprotein is produced under the assay conditions), and/or a high specific activity.

The D-Amino-Acid Oxidase may have a high specific activity, which can be of at least 20, in particular at least 40, or even at least 55 units/mg for at least one, or only one, specific D-amino acids. More precisely, the D-Amino-Acid Oxidase may have a high specific activity, which can be of at least 20, in particular at least 40, or even at least 55 units/mg D-serine and/or least 50, in particular at least 75, or even at least 100 units/mg for D-alanine. One DAAO unit is defined as the amount of enzyme that converts 1 μmole of D-alanine per minute at 25° C.

The means for oxidation of the D-amino-acid can be any type of D-amino-acid oxidase (DAAO), notably non mammalian, in particular a DAAO from a yeast or a microorganism. The DAAO may be chosen from the group comprising Rhodotorula gracilis DAAO (RgDAAO), Trigonopsis variabilis DAAO, V. luteoalbum DAAO, F. oxysporum DAAO and derivatives thereof.

By “derivatives” is meant in the instant invention, an enzyme having at least 65%, in particular at least 75%, more particularly at least 85%, notably at least 90%, even at least 95%, even more particularly at least 99 % identity of the amino acids with the corresponding enzyme. The derivatives have the catalitic activity, for example a D-amino acid oxidase, in particular with an efficiency of the same order, i.e. at least 50%, more specially at least 75%, in particular at least 100%, even more than 150% of the efficiency of the original enzyme. The efficiency of the enzyme may be defined as the ratio k_{cat}/Kₘ, assayed with an oxygen electrode at pH 8.5 and 25° C., at air oxygen saturation ([O₂]=0.253 mM) [G. Molla, C. Vegezzi, M. S. Pilone, L. Pollegioni, Overexpression in Escherichia coli of a recombinant chimeric Rhodotorula gracilis D-amino acid oxidase, Prot. Express. Purif., 14 (1998) 289-294]. [2]

The D-amino acids for which the concentration is measured is a substrate of the D-Amino-Acid Oxidase (DAAO). The D-amino acids may be a natural or a synthetic D-amino acid. It may be an alpha, beta, gamma or omega D-amino acid.

The amino acids may be selected from the group of D-alanine, D-valine, D-leucine, D-isoleucine, D-methionine, D-proline, D-phenylalanine, D-tryptophan, D-serine, D-threonine, D-tyrosine, D-cysteine, D-asparagine, D-glutamine, D-lysine, D-arginine and D-histidine, in particular D-serine, and their derivatives.

The means for oxidation of the D-amino-acids may be immobilized on the electrode by:

Covalent immobilization, for example
by using bifunctional agents, such as glutaraldehyde:
   a) by using vapors of the bifunctional agent, or
   b) by preliminary mixing solution of bifunctional agents with enzymatic mixture

Entrapping D-Amino-Acid Oxidase (DAAO), for example into a reticular matrix, which can be formed by different types of polymeric reactions, in particular stimulated by UV, irradiation or chemical catalyst, or electrogenerated, like polymers of phenylenediamine, pyrrole or aniline,

Immobilisation into the body of the electrode, such as paste type electrode, for example mix of enzyme, graphite powder, mediator, and/or

Adsorption, in particular on a high surface layered material, such as cellulose acetate, nylon, inorganic gels (for example silica gel) and such types of membranes.

The means for optimising the detection of compound B may correspond to means for the catalysis of hydrogen peroxide oxidation.

The means for optimising the detection of said compound B may be selected from:
an electrochemical pre-treatment of the electrode, in particular carbon based electrode, and
deposition, for example via electrochemical or vacuum deposition, of one or several metals, such as metals catalysing the oxidation of compound B,
deposition of an artificial system catalysing the oxidation of compound B, such as artificial peroxidase system, in particular based on Prussian Blue,
Horseradish peroxidase (HRP), and
use of nanoparticles, for example of Gold, Platinum, Silicon oxides, Zinc, carbon nanotubes, etc.

In other words, the means for optimising the detection of said compound B may be selected from:
an electrochemical pre-treatment of the electrode,
deposition of at least one metal
deposition of an artificial system catalysing the oxidation of compound B,
Horse Radish peroxidase (HRP),
use of nanoparticles, and
a combination thereof.

When the means for optimising the detection of said compound B is a metal it can be selected from the noble metal group comprising platinum, gold, ruthenium, rhodium, palladium, iridium, osmium, and from other metals like iron, chromium, nickel and tungsten.

The means for reducing interferences may be limiting oxidation of compounds other than compound B which may be oxidised by the electrode, in particular by limiting the access and/or the contact of these compounds with the electrode, in other words in particular by limiting the access and/or the contact of said compound other than the compound B with the electrode.

The means for reducing interferences may be selected from the group comprising:

electrogenerated polymeric membranes, for example based on monomers like benzene and its derivatives, such as phenylenediamine, resorcinol, phenol; naphthalene and its derivatives; pyrrole and its derivatives; aniline and its derivatives; and combinations thereof,

membranes deposited from solutions of polymers, charged, like Nafion, or neutral, like polyurethane and polyvinyl chloride, or derivatives of cellulose,

non polymeric charged molecule layer, for example such a layer is deposited via solution, such as phospholipids and fatty acids, like stearic acid,

use of at least an enzyme that degrades interfering molecule(s), for example ascorbic acid oxidase, in particular this enzyme may be immobilized on the electrode, and

use of electrochemical methods allowing to discriminate between different molecules, for example pulsed amperometry, cyclic voltammetry or pulsed voltammetry.

The means for reducing interferences may be a layer of a compound selected from the group comprising, poly-meta-, para- or ortho-phenylenediamine (PPD), substituted naphthalene-based polymers, rejecting membrane or a rejecting polymer membrane.

Among the electrode materials that can be used the following may be cited:

electrodes based on platinum, rhodium, palladium, gold, ruthenium, possibly under an alloy form, for example with gold, iridium or other noble metals, among such alloys an example is platinum 90% and iridium 10%

electrodes based on carbon materials, graphite (rods), glassy carbon (rods), carbon fibres, diamond,

electrodes covered by nanoparticles of noble metals, carbon materials or a mixture thereof, and

ceramic based amperometric electrodes.

Some type of electrode materials may be excluded as they possibly lead to inactivation of the D-Amino-Acid Oxidase (DAAO), in particular Ag and Hg electrodes.

The electrode shape may be a disk, a ring, a rod, a cylinder, a cone or a hemisphere.

Following a specific embodiment, the microelectrode comprises, or consists of, a carbon fibre with the working part covered by a layer of ruthenium, a layer of PPD and a layer of DAAO.

Following another aspect, the subject matter of the invention is a device for detecting and/or measuring the concentration of a D-amino-acid in a medium, in particular in vivo, comprising an electrode as defined above.

This device may further comprise an acquisition card driving an amplifier, in particular equipped with a two or three electrodes potentiostat.

This device may also comprise a working electrode and a reference electrode for the two-electrode system, and an additional auxiliary or counter electrode for the three-electrode system.

Following another aspect, the subject matter of the invention is a method for detecting and/or measuring the concentration of a D-amino-acid in a medium, in particular in vivo, comprising the following steps:

a working microelectrode of the present invention is placed in said medium,

a working potential is applied, in particular by cyclic voltammetry, pulsed voltammetry/amperometry or by applying a constant potential.

The potential may be fixed between −1 and +1 V vs. Ag/AgCl for the detection of peroxide (H₂O₂).

An efficient detection method is the continuous amperometry at a fixed potential of +0.55 V vs. Ag/AgCl. In this case the peroxide oxidation is seen via a jump of current at the level of working electrode (FIG. 1). The relationship between oxidation and peroxide concentration in the solution is linear, in particular in a range of 0.01 μM to 10 μM (FIG. 2).

Following another aspect, a subject matter of the invention is a method for making a microelectrode comprising the following steps:

treating the electrode in order to increase its sensitivity to compound B, in particular via a metallization, for example an electrodeposition of a metal,

the deposition of a polymer film in order to discriminate compound B from other compounds, i.e. other compounds present in the medium and, which may be oxidised by the electrode,

the deposition of a film of an enzyme oxidising

D-amino-acid to compound B on the electrode, and then immobilizing said enzyme, in particular via glutaraldehyde (vapours or solution).

The enzyme may be immobilised via a cross-linking process, for example with an aldehyde, such as glutaraldehyde, or with other molecules such as poly(ethylene glycol) 400 diglycidyl ester (PEGDGE) or may be imprisoned in the Material of the electrode, such as the carbon paste. It can also be entrapped or encapsulated in a polymer film, such as polypyrrole.

The metal covering the working part of the electrode may be deposited via classical methods known from one skilled in the art, for example by a gel impregnated with the metal, for example the osmium hydrogel.

### Examples

### Example 1

### Preparation of a Ruthenium-Coated Carbon Fibre Microelectrode for Measuring D-serine Concentration

1. Preparation of the Carbon Fibre Electrode

A 7 μm diameter carbon fibre electrode (Goodfellow Cambridge Ltd., Huntington, UK) glued to a copper wire by a silver paint (Radiospares, Beauvais, FR) deposition. The wire is placed in a glass micropipette on which the extremity is cut in order to allow the carbon fibre to protrude. The junction between glass micropipette and the carbon fibre is sealed by an epoxy resin, in order to insure its tightness, and the carbon fibre is cut at 150 μm.

The electrode is then put into ethanol for 20 minutes to clean the fibre of its impurities.

2. Electrodeposition of Ruthenium

The electrodeposition of ruthenium has been done by applying for 20 minutes a potential of −450 mV Vs Ag/AgCl to the carbon fibre electrode which is in a solution of RuCl₃ at a concentration of 100 μg/ml and pH 2.5 (ruthenium atomic absorption standard solution, Sigma, Saint Quentin, France).

After the deposition of Ruthenium, the electrode has been cycled 15 times between 0 and 700 mV for stabilisation.

3. Deposition of a PPD Film Layer

The deposition has been done electrochemically by putting the electrode 20 minutes in a solution of 100 mM of m-PD and applying a potential of +700 mV vs. Ag/AgCl.

4. Deposition of a D-amino-acid Oxidase (DAAO) Film

A film of enzyme is deposited on the electrode by dipping the electrode into an enzyme solution comprising 56 mg/ml of RgDAAO, 25 mg/ml Bovine Serum Albumine (BSA) and 1% glycerol. The electrode is removed from the solution and then the enzyme is immobilised by exposition to vapours of a 50% glutaraldehyde solution.

Alternatively the enzyme may be immobilized by using a solution of enzyme comprising glutaraldehyde 0.17%; glycerol 2%; RgDAAO 37 mg/ml; BSA 17 mg/ml.

The thickness of the DAAO film obtained is about 15 to 25 μm.

### Example 2

### Preparation of a Platinum Disk-Shaped Electrode Covered with Yeast D-amino Acid Oxidase for D-serine Detection

Electrodes were prepared by inserting a 400 μm diameter platinum wire into a glass capillary and sealing the tip of the pipette by melting the glass over a flame. The platinum wire inside the pipette was then soldered to a copper wire using Pb—Sn solder and the back end of the pipette was sealed using epoxy resin. The tip of the electrode was then polished using sand paper of increasing fineness (finishing with 0.3 μm Al₂O₃ emery paste) in order to expose a clean 400 μm diameter Pt disk.

A first layer of poly-m-phenylenediamine was deposited by electropolymerization of m-phenylenediamine (see example 1). The enzymatic layer was deposited by laying a small drop (˜30 nl) of a solution of Rhodotorula Gracilis D-amino Acid Oxidase RgDAAO (56 mg/ml of RgDAAO, 25 mg/ml Bovine Serum Albumine (BSA) and 1% glycerol) onto the Pt disk and exposing it to saturated glutaraldehyde vapors for 5 min. The electrode was then rinsed in Phosphate Buffer Saline (PBS), dried and stored at 4° C. in dry atmosphere. The thickness of the resulting membrane was about 5-10 μm.

### Example 3

### D-serine Detection Using the Microelectrode of the Present Invention

The microelectrode used in this example was the same as in example 2.

A calibration curve in D-serine ranging from 5 μM to 1.2 mM was done with the microelectrode of the example 2 (FIG. 3B). The microelectrode's response is linear with concentration between 5 μM and at least 100 μM.

The oxidation current at the microelectrode upon application of serotonin (5-HT, 20 μM), hydrogen peroxide (H₂O₂, 1 μM), and D-serine (1 μM and 2 μM) was measured (FIG. 3A). D-serine and H₂O₂ were detected as a step in oxidation current, and interference from 5-HT was rejected (FIG. 3B).

### Example 4

### Devices for Measuring the Concentration of D-serine

The system of command and acquisition is composed of an acquisition card ITC-18 (Instrutech Corporation, Greatneck, N.Y., USA), driving an amplifier VA-10 (NPI Electronics, Tamm, Germany) equipped with a two-electrode (working electrode corresponding to the microelectrode of the invention and home made reference electrode composed of a chlorided silver wire) potentiostat. The acquisition card is driven with the SVoltare software.

### Example 5

### Cell Cultures

Primary cultures of cortical astrocytes were prepared from newborn rats and cultured until reaching confluence (14 days). The astrocytes were then treated 5 days with 8 μM cytosine arabinofuranoside in order to eliminate remaining microglia.

The cells were placed in a chamber perfused with Krebs buffer. The microelectrode of the invention has been set on top of the cells without touching them. Triton X-100 1% or digitonin 50 μM was added in the perfusion medium to disrupt the integrity of the cellular membrane. This treatment induced an increase in oxidation current at the microelectrode which is due to the release of the intracellular stores of D-serine (FIG. 4). An estimation of the intracellular stores of D-serine can be given by measuring the integrated surface of the oxidation current. This method allows therefore the measurement of the intracellular stores of D-serine of cells in culture.

### Example 6

### In Vivo Recordings in the CNS of an Anesthetized Rat

Male Wistar rats weighing between 300 and 400 g were anesthesized with chloral hydrate and placed in a stereotaxic apparatus. The reference electrode was laid on the surface of the skull and the working electrode was inserted in the cerebellum at about 1.5 mm under the pial surface.

After a 30 min stabilization time, the oxidation current at the working electrode under a potential of 0.5V vs. Ag/AgCl was recorded.

D-serine was then injected intraperitoneally at a concentration of 1 g/kg of body weight.

After a few minutes the signal by our method was increased by about 90 pA, suggesting that D-serine penetrating through the blood-brain barrier into the central nervous system was detected by the microelectrode as defined in example 1.

### Example 7

### D-serine Detection in Rat Brain Extracts

### A. Preparation of Brain Samples

Male Wistar rats (300-400 g) were decapitated under isofurane anesthesia, and the forebrain was removed and homogenized in 5 ml of 5% trichloroacetic acid (TCA) to precipitate proteins. The homogenate was then centrifuged at 20 000 g for 10 min. TCA was extracted from the supernatant using ether, before lyophilization and storage at −20° C.

### B. High Pressure Liquid Chromatography (HPLC) Measurements

Lyophilized brain extracts were dissolved in 1 ml deionized water, and 50 μl were derivatized with 0.8 mg N-acetyl-cysteine and 0.25 mg o-phthaldialdehyde in a 0.1M borate buffer (pH 10.4). HPLC measurements were performed using a Waters Alliance instrument (Waters Corporation, Guyancourt, France) with a Waters symmetry column (4.6×250 mm). The column and sample compartments were kept at 30 and 4° C. respectively. Flow rate was set at 1 ml/min and run time was 25 min for all analyses. L- and D-serine were detected with an isocratic method using mobile phase A (990 ml of 0.1 M sodium acetate and 10 ml tetrahydrofurane, pH 6.2) and the column was washed using mobile phase B (500 ml of 0.1 M sodium acetate, 470 ml acetonitrile, 30 ml tetrahydrofurane, pH 6.2). Amino acids derivatives were detected using a Waters fluorescence detector (excitation 344 nm, emission 443 nm), and data were acquired using the Empower Pro software package (Waters Corporation, Guyancourt, France). Calibration of D-serine detection was performed using a 7-point standard curve.

### C. Comparison Between D-serine Detection Using HPLC or the Microelectrode of the Present Invention.

D-serine concentration in brain extracts was estimated by two independent methods, using HPLC or the RgDAAO microelectrode (FIG. 5). HPLC estimated D-serine concentration to be about 130 μM whereas the microelectrode yielded an estimate of 139 μM. In addition, we preincubated the brain extracts with DAAO and catalase to eliminate D-amino acids and peroxide present in the medium (FIG. 5A and FIG. 5B). HPLC measurements indicated that D-serine had completely disappeared in the brain extract, whereas the microelectrode detected only a small amperometric response that amounted to about 5% of the original response before the enzymatic treatment (FIG. 5C).

These data indicate that the microelectrode of the present invention can selectively detect D-serine in complex biological media such as brain extracts, with no more than 5-7% deviation from HPLC measurements.

### REFERENCES

[1] Stulik K, Amatore C, Holub K, Marecek V and Kutner W (2000) Microelectrodes. Definitions, characterization and applications (technical report). Pure Appl. Chem. 72: 1483-92.
[2] G. Molla, C. Vegezzi, M. S. Pilone, L. Pollegioni, Overexpression in Escherichia coil of a recombinant chimeric Rhodotorula gracilis D-amino acid oxidase, Prot. Express. Purif., 14 (1998) 289-294

1. Microelectrode for measuring the concentration of a D-amino acid, said microelectrode comprising:
mean for oxidation of said D-amino acid into at least a compound B, said means for oxidation including at least one nonmammalian D-amino acid oxidase (DAAO),
means for optimising the detection of said compound B, and means for reducing interferences due to the oxidation of other species than compound B.

2. Microelectrode according to claim 1, wherein the said D-amino-acid oxidase leads to the production of hydrogen peroxide as compound B.

3. Microelectrode according to claim 1, wherein the D-amino-acid oxidase (DAAO) does not need the addition of a cofactor in the medium to oxidise the D-amino-acid and/or shows a high specific activity.

4. Microelectrode according to claim 1, wherein the DAAO is from a yeast or a microorganism.

5. Microelectrode according to claim 4 wherein, the DAAO is selected from the group comprising Rhodotorula gracilis DAAO (RgDAAO), Trigonopsis variabilis DAAO, V. luteoalbum DAAO, F. oxysporum DAAO and derivatives thereof.

6. Microelectrode according to claim 1, wherein the D-amino acid is selected from the group comprising D-alanine, D-valine, D-leucine, D-isoleucine, D-methionine, D-proline, D-phenylalanine, D-tryptcphan, D-serine, D-threonine, D-tyrosine, D-cysteine,D-asparagine, D-glutamine, D-lysine, D-arginine and D-histidine, and derivatives thereof.

7. Microelectrode according to claim 6, wherein the D-amino acid is D-serine.

8. Microelectrode according to claim 1, wherein the means for oxidation of the D-amino-acid is immobilized on the electrode by:
Covalent immobilization,
Entrapping D-Amino-Acid Oxidase (DAAO),
Immobilisation into the body of the electrode, and/or
Adsorption.

9. Microelectrode according to claim 1, wherein the means for optimising the detection of .said compound B is selected from:
an electrochemical pre-treatment of the electrode,
deposition of at least one metal
deposition of an artificial system catalysing the oxidation of compound B,
Horse Radish peroxidase (HRP),
use of nanoparticles, and
a combination thereof.

10. Microelectrode according claim 9 wherein
said electrode is a carbon based electrode,
said deposition is a electrochemical or vacuum deposition of a metal catalysing the oxidation of compound B,
said artificial system is an artificial peroxidase system based on Prussian Blue.

11. Microelectrode according to claim 9, wherein the metal is selected from noble metal group comprising platinum, gold, ruthenium, rhodium, palladium, iridium, osmium, and from other metals like iron, chromium, nickel, tungsten.

12. Microelectrode according to claim 1, wherein the means for reducing interferences are limiting oxidation of compounds other than compound B which may be oxidised by the electrode.

13. Microelectrode according claim 12, wherein the means for reducing interferences is done by limiting the access and/or the contact of said compounds other than compound B with the electrode.

14. Microelectrode according to claim 1, wherein the means for reducing interferences are selected from:
electrogenerated polymeric membranes,
membranes deposited from solutions of charged or neutral polymers,
non polymeric charged molecule layer,
use of at least an enzyme that degrades interfering molecule(s), and
use of electrochemical methods allowing to discriminate between different molecules.

15. Microelectrode according to claim 1, wherein the electrode is chosen from the group comprising:
electrodes based on platinum, rhodium, palladium, gold, ruthenium
electrodes based on carbon materials, graphite and glossy carbon, carbon fibres, diamond,
electrodes covered by nanoparticles based on noble metals, carbon materials or a mixture thereof, and
ceramic based amperometric electrodes.

16. Microelectrode according to claim 1, wherein the microelectrode is a carbon fibre electrode covered by a layer of ruthenium, a layer of PPD and a layer of D-Amino-Acid Oxidase (DAAO).

17. Device for detecting and/or measuring the concentration of a D-amino-acid in a medium, comprising an electrode according to any one of claims 1 to 16.

18. Method for detecting and/or measuring the concentration of a D-amino-acid in a medium comprising the following steps:
placing a working microelectrode according to any of claims 1 to 16 in said medium, and
applying a working potential.

19. Method according claim 18 wherein the working potential is applied by cyclic voltammetry, pulsed voltammetry/amperometry or by applying a constant potential.

20. Method for making a microelectrode comprising the following steps:
treating the electrode in order to increase its sensitivity to compound B,—the deposition of a polymer film in order to discriminate compound B from other compounds present in the medium and which may be oxidised by the electrode,
the deposition of a film of an enzyme oxidising compound A to compound B on the electrode, and then immobilizing said enzyme.

21. Method according claim 20, wherein the electrode is treated via a metallization.
